(19) United States
(12) Patent Application Publication
(10) Pub. No.: US 2008/0187572 A1
(43) Pub. Date: Aug. 7, 2008

(54) Matrix Metalloproteinase Inhibitors of Tgfb-Induced Subcapsular Cataract Formation

(75) Inventor: Judith West-Mays, Oakville (CA)

Correspondence Address: OCCHIUTI ROHLICEK & TSAO, LLP, 10 FAWCETT STREET, CAMBRIDGE, MA, 02138, US

(73) Assignee: McMaster University, Hamilton,Ontario (CA)

(21) Appl. No.: 11/912,729

(22) PCT Filed: Apr. 28, 2006
(86) PCT No.: PCT/CA2006/000661

§ 371 Date: Mar. 11, 2008

Related U.S. Application Data
(60) Provisional application No. 60/675,443, filed on Apr. 28, 2005.

(51) Int. Cl. A61K 9/00; A61K 31/195; A61P 27/12

(52) U.S. Cl. 424/429; 514/563

(57) ABSTRACT

The present invention provides novel methods and compositions for the treatment and/or prevention of cataracts. A composition that inhibits destabilization of E-cadherin is administered. Preferably the composition comprises a matrix metalloproteinase inhibitor.

### FIELD OF INVENTION

The present invention relates to methods, compositions and devices for the prevention or treatment of cataracts.

### BACKGROUND OF THE INVENTION

Cataract is a refractory ocular disease leads to lower vision and, in some cases, to blindness. As the crystalline lens loses its normal transparency, less light passes through the lens. The degree to which vision is lost depends on the degree of opacification of the lens.

Many different factors can lead to cataract formation. These include aging, congenital lesions or trauma, some medicines such as steroids and glaucoma medications, cigarettes, and alcohol. Inborn metabolic errors such as galactosemia and diseases like diabetes, atopic dermatitis and retinitis pigmentosa are also associated with cataract formation.

The lens is a relatively simple tissue composed of two cell types, epithelial cells and fiber cells. In adults, lens proliferation and differentiation occurs near the lens equator. However, in a pathological situation, the anterior lens epithelial cells (LECs) can be triggered to proliferate and multilayer beneath the lens capsule. This can trigger further changes that lead to the development of cataracts. Fibrous anterior subcapsular cataract (ASC) plaques are formed that develop into distinct opacities in the lens.

Cataracts are a major problem in the aging population. It is estimated that about 60-70% of people in their sixties and nearly 100% of people over eighty have some degree of cataract and cataract excision is the most common type of operation in the aged population. While cataract surgery is highly effective, the incidence of secondary cataract after surgery is a problem. Secondary cataract results in opacity on the surface of the remaining posterior capsule following extracapsular cataract extraction. Secondary cataract results from migration and proliferation of residual lens epithelial cells, which are not completely removed at the time of extraction of the lens cortex, onto the posterior capsule leading to posterior capsule opacification. Secondary cataract is also associated with abnormal proliferation of the residual lens epithelial cells in the equator followed by formation of Elschnig pearls. In cataract surgery, it is impossible to remove lens epithelial cells completely, and consequently it is difficult to always prevent secondary cataract. Secondary cataract, also known as posterior capsular opacification (PCO), is very similar to ASC.

While various surgical techniques have been developed for the treatment of cataracts, it is desirable to avoid surgery if possible. It is also desirable to prevent or slow down the development of cataracts and also to avoid the development of secondary cataracts post-operatively. Several attempts have been made in the field of cataract medication to find compositions that can be used to treat or prevent the formation of cataracts.

U.S. Pat. No. 6,914,057 discloses a method of reducing the risk of cataract development that comprises administering a tetracycline derivative. The tetracycline derivative is preferably administered systemically or orally.

U.S. Pat. No. 5,925,617 discloses a prophylactic/therapeutic composition for secondary cataract. The method comprises administering a composition comprising a polypeptide that inhibits cell adhesion and a lactic acid/glycolic acid polymer.

U.S. Pat. No. 5,876,438 discloses a device for the intraocular delivery of immunotoxins for the prevention of secondary cataract. The immunotoxins inhibit the proliferation of lens epithelial cells.

U.S. Pat. No. 5,874,455 discloses a method for the treatment of cataract that comprises administering an effective amount of a radical scavenger such as a thiol derivative or a disulfide derivative.

U.S. Pat. No. 5,827,862 discloses an agent for the prophylaxis or treatment of cataract comprising a carbostyril compound.

U.S. Pat. No. 5,698,585 discloses that 3(2H)— furanone derivatives or their salts can be used to prevent or treat cataract.

In U.S. Pat. No. 5,696,091, the intraocular use of combinations of lens epithelia) cell growth stimulators (e.g., TGF-.beta.) and antimetabolites (e.g., mitomycin C) is described. The combination is applied to the capsular bag to prevent or retard the formation of secondary cataracts following cataract surgery. The lens epithelial cell stimulators activate DNA synthesis in dormant lens epithelial cells, and thereby make those cells, susceptible to the anti-metabolites. This enables the antimetabolites to suppress the proliferation of lens epithelial cells to a much greater extent, relative to the proliferation observed ken the metabolites alone are utilized. The Increased suppression of the growth of lens epithelial cells results in a significant improvement in the ability to prevent or retard the formation of opacities on the lens capsule (i.e., secondary cataracts).

U.S. Pat. No. 5,686,487 discloses a method for treatment of a cataract that comprises administering, to a subject in need of such treatment, a ketoprostaglandin compound in an amount effective in treatment of cataract.

While many efforts have been made to develop prophylactic or therapeutic agents for cataracts, there remained a need for further elucidation of the mechanisms involved in cataract formation and agents that interfere with those mechanisms. In our society where the average age of the population is increasing and the elderly are expecting a better quality of life than in previous generations, the prevention and treatment of cataract is becoming more and more critical. Currently, the standard treatment of cataract involves the correction of vision using eyeglasses, contact lenses etc. or surgical operations such as extracapsular cataract extraction and insertion of an intraocular lens. However, at the present time, there are no proven therapeutic agents that have been shown to inhibit the development of cataract. Thus, there has been a real need for the development of an effective therapeutic agent for the treatment and/or prevention of cataract.

### SUMMARY OF THE INVENTION

The present invention addresses the need for novel therapeutic strategies to prevent or treat cataracts.

In one aspect of the invention, a method of treating or preventing cataracts is provided. The method comprises administering to a subject in need of such treatment a therapeutically effective amount of an agent that prevents E-cadherin destabilization. In a preferred embodiment, the agent that prevents E-cadherin destabilization is a matrix metalloproteinase inhibitor. More preferably, the matrix metalloproteinase inhibitor (MMPI) is an MMP-2/9 specific inhibitor. Broadly active MMPIs are also preferred agents. In one preferred embodiment, the inhibitor is an Ilomostat (GM6001). In another preferred embodiment, the inhibitor is (2R)-[(4 Biphenylylsulfonyl)amino]-N-hydroxyl-3-phenylpropionamide).

In a preferred aspect of the invention, the inhibitor is locally administered. The inhibitor may be delivered via a controlled release device. Examples of controlled release devices include, but are not limited to, coated contact lenses and intra-ocular lenses. In one embodiment, the controlled release device is implanted in the eye.

In another aspect of the invention, the inhibitor is delivered to the site by injection.

In another aspect of the invention, the inhibitor is administered as eye drops.

In the method of the present invention, the inhibitor is preferably administered in a range of about 1 μg/ml to about 500 μg/ml, preferably 1 μg/ml to 250 μg/ml, more preferably 1 μg/ml to 100 μg/ml, most preferably 5 μg/ml to 25 μg/ml.

In a preferred embodiment of the method of the present invention the inhibitor may be administered in combination with an additional MMP inhibitor. In another embodiment, the inhibitor may be administered with another pharmacologically active agent.

In another aspect of the invention a composition for the treatment or prevention of cataract comprising an MMPI and a pharmaceutically acceptable carrier is provided. The MMPI is preferably selected from the group consisting of: ((2R)-[(4-Biphenylylsulfonyl)amino]-N-hydroxyl-3-phenylpropionamide); actinonin (3-[[1-[[2-hydroxymethyl-1-pyrolidinyl]carbamoyl]-octano-hydroxamic acid); bromocyclic-adenosine monophosphate; N-chlorotaurine; BATIMISTAT (BB-94); CT1166 (N1{N-[2-(morpholinosulphonylamino)-ethyl]-3-cyclohexyl-2-(S)-propanamidyl}-N4-hydroxy-2-(R)-[3-(4-methylphenyl)propyl]-succinamide); estramustine (estradiol-3-bis(2-chloroethyl)carbamat-e); eicosa-pentaenoic acid; MARIMASTAT (BB-2516); matiystatin-B; peptidyl hydroxamic acids; N-phosphonalkyl dipeptides; protocatechuic aldehyde (3,4-dihydroxybenzaldehyde); Ro-31-7467 (2-[(5-bromo-2,3-dihydro-6-hydroxy-1,3-dioxo-1H-benz[de]isoquinol-In-2-yl)methyl](hydroxy)-[phosphinyl]-N-(2-oxo-3-azacyclotridecanyl)-4-met-hylvaleramide); tetracyclines; 1,10-phenanthroline (o-phenanthroline[4-(N-hydroxyamino)-2R-isobutyl-3S-(thiopen-2-ylthiomethyl)-succinyl]-L-p-henylalanin-N-methylamidecarboxyalkylamino-based compounds; chelators (EDTA, cysteine, acetylcysteine, D-penicillamine, and gold salts); bis(dioxopiperzaine); NEOVASTAT; KB-R7785; ILOMASTAT; RPR-122818; SOLIMASTAT; BB-1101; BB-2983; BB3644; BMS-275291; D-1927, D-5410; CH-5902, CH-138; CMT-3; DERMOSTAT; DAC-MMPI; RS-1130830 and RS-113-080; GM-1339; GI-155704A; ONO-4817 AG-3433, AG-3088, PRINOMASTAT; CP-544439; POL-641: SC-964; S[)-2590; PNU-142769; SU-5402; PGE-2946979, PGE4304887; fibrolase-conjugate; EF-13; S-3304; CGS-25015 and CGS-27023A; XR-168; RO1130830: D-9120; BB2827; BB-1101 (2S-allyl-N1-hydroxy-3R-isobutyl-N4-(1 S-methylcarbamoyl-2-phenylethyl)-su-ccinamide), BB-2983, solimastat (N′-[2,2-Dimethyl-1 (S)-[N-(2-pyridyl)carba-moyl]propyl]-N4-hydroxy-2(R)-isobutyl-3(S)-methoxysuccinamide), N4-hydroxy-N1-[2-(methylamino)-2-oxo-1-(phenylmethyl)ethyl]-2-(2-methylpr-opyl)-3-[(2-thienylthio)methyl]-, [2R-[1 (S*),2R*,3 S*]]-[CAS]), rebimastat (L-Valinamide, N—((2S)-2-mercapto-1-oxo-4-(3,4,4-trimethyl-2,5-dioxo-1-imidazolidinyl)butyl)-L-leucyl-N,3-dimethyl-[CAS]); PS-508; CH-715; nimesulide (Methanesulfonamide, N-(4-nitro-2-phenoxyphenyl)-[CAS]-), hexahydro-2-[2(R)-[1 (RS-(hydroxycarbamoyl)-4-phenylbutyl]nonanoyl]-N—(-2,2,6,6-etramethyl-4-piperndinyl)-3(S)-pyridazine carboxamide, Cipemastat(1-Piperidinebutanamide, .beta.-(cyclopentylmethyl)-N-hydroxy-Gamma-oxo-A-lpha-[(3,4,4-trimethyl-2,5-dioxo-1-imidazolidinyl)methyl]-,(AlphaR,.beta.R-)-[CAS]), 5-(4′-biphenyl)-5-[N-(4-nitrophenyl)piperazinyl]barbituric acid, 6-methoxy-1,2,3,4-tetrahydro-norharman-1-carboxylic acid, Ro-31-4724 (L-Alanine, N-[2-[2-(hydroxyamino)-2-oxoethyl]-4-methyl-1-oxopentyl]-L-le-ucyl-, ethyl ester[CAS]), N-hydroxy-2,2-dimethyl-4-((4-(4-pyridinyloxy)phe-nyl)sulfonyl)-, (3R)-[CAS]), PNU-142769 (2H-Isoindole-2-butanamide, 1,3-dihydro-N-hydroxy-Alpha-[(3S)-3-(2-methylpropyl)-2-oxo-1-(2-phenyleth-yl)-3-pyrrolidinyl]-1,3-dioxo-, (AlphaR)-[CAS]), (S)-1-[2-[[[(4,5-Dihydro-5-thioxo-1,3,4-thiadiazol-2-yl)amino]-carbonyl]amino]-1-oxo-3-(pentafluoro-phenyl)propyl]-4-(2-pyridinyl)piperazine, SC-77964, PNU-171829, N-hydroxy-2(R)-[(4-methoxybenzene-sulfonyl)(4-picolyl)amino]-2-(2-tetrahy-drofuranyl)-acetamide, L-758364 ((1,1′-Biphenyl)-4-hexanoic acid, Alpha-butyl-Gamma-(((2,2-dimethyl-1-((methylamino)carbonyl)propyl)amino)c-arbonyl)-4′-fluoro-, (AlphaS-(AphaR*,GammaS*(R*)))-[CAS]); antibodies; and analogues or derivatives thereof.

In a preferred embodiment the MMPI is selected from the group consisting of an MMP-2 inhibitor, an MMP-9 inhibitor and an MMP-2/9 inhibitor. In another preferred embodiment, the MMPI is Ilomastat (GM6001) or ((2R)-[(4-Biphenylylsulfonyl)amino]-N-hydroxyl-3-phenylpropionamide).

In another aspect of the invention, the use of a matrix metalloproteinase inhibitor in the manufacture of the medicament for the treatment or prevention of cataracts is provided. In a preferred embodiment, the medicament is prepared for the treatment or prevention of subcapsular cataracts, more preferably anterior subcapsular cataracts and secondary cataracts.

In a further aspect of the invention, a delivery device for the administration of a matrix metalloproteinase inhibitor to a region of an eye of a patient is provided. In one preferred embodiment, the delivery system is adapted to be inserted into the eye. Devices of this type include coated contact lenses, coated intra-ocular lenses and eye drops. In another preferred embodiment, a delivery system is implanted into the eye. In another preferred embodiment, the delivery system provides for controlled release of the inhibitor.

It is an object of one aspect of the invention to provide matrix metalloproteinase inhibitors of subcapsular cataract formation and methods of using same.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1A illustrates an optical scan of a control lens;

FIG. 1B illustrates an optical scan of a TGFβ treated lens;

FIG. 2 illustrates graphically the back vertex variability for lenses that are untreated, treated with TGFβ, or treated with TGFβ and GM6001;

FIG. 3 is a bar graph illustrating the dosage effect of GM6001 on TGFβ induced BVD variability:

FIG. 4A illustrates a control lens;

FIG. 4B illustrates a lens treated with TGFβ;

FIG. 4C illustrates a lens co-treated with TGFβ and GM6001;

FIG. 4D illustrates a lens cotreated with TGFβ and MMPI-2/9;

FIG. 4E illustrates a section of a control lens;

FIG. 4F illustrates a section of a lens treated with TGFβ;

FIG. 4G illustrates a section of a lens co-treated with TGFβ and GM6001;

FIG. 4H illustrates a section of a lens cotreated with TGFβ and MMPI-2/9;

FIGS. 5A, B and C illustrate the effect of TGFβ and MMPIs on MMP expression;

FIGS. 6A, B and C demonstrate the effect of TGFβ and MMPIs on E-cadherin mRNA and destabilization; and

FIG. 7 illustrates cross-sections of treated and untreated eyes.

### DETAILED DESCRIPTION

Cataracts are a leading cause of blindness worldwide, yet there is currently no pharmacological agents on the market that prevent or inhibit the progression of cataract formation. The present invention provides novel pharmaceutical compositions, devices, and methods for the treatment and prevention of cataracts.

In one aspect of the Invention, a novel therapeutic for the treatment of fibrotic pathologies, including anterior subcapsular cataracts (ASCs) is provided. ASC formation involves increased proliferation and transition of lens epithelial cells into myofibroblasts, through epithelial-mesenchymal transformation that results in opaque plaques beneath the lens capsule.

TGF-β plays an important role in the development of ASCs. A TGF-β induced rat cataract model was used to determine the role of matrix metalloproteinases (MMPs) in ASC formation. Matrix Metalloproteinases (MMPs) are a family of zinc endopeptidases that art as key regulators of tissue remodeling and have been shown to participate in a number of ocular diseases including retinal disease, glaucoma, and corneal disorders. Using this model, a novel method for the treatment or prevention of cataracts was demonstrated. Treatment of rat lenses with TGFβ results in enhanced secretion of MMP2 and MMP9. Treatment with MMP inhibitors suppresses the TGFβ-induced subcapsular changes including the epithelial-to-mesenchymal transition (EMT) of lens epithelial cells (LECs). It was also demonstrated that lenses treated with TGFβ exhibited a 70 kDa E-cadherin fragment indicative of E-cadherin destabilization. This is accompanied by attenuated levels of E-cadherin mRNA versus controls. Further details on the experiments can be found in the examples below and in the attached figures. The findings demonstrate that E-cadherin destabilization in the lens is associated with cataract formation. This may be mediated by MMPs and suppression of this phenomenon through the use of MMP is inhibits plaque formation that leads to cataracts. The rat lens model is a well-established model of cataract formation in the human eye. Demonstration of a therapeutic affect in this model is indicative of an effective therapy for treatment of the human cataract. Since cataracts have been shown to form in this model in the same manner as they form in the human lens in vivo, identification of and interruption of mechanisms of pathogenesis in this model can be translated into therapies for in vivo human and/or animal cataract prevention and treatment.

Cataract formation has been demonstrated to be associated with E-cadherin destabilization. Thus, the invention provides a method for preventing the formation of cataracts or inhibiting the progression of a cataract by administering an agent that reduces or eliminates the E-cadherin destabilization that is associated with cataract formation. A composition that stabilizes E-cadherin is also provided for the treatment of cataract. A device incorporating an agent that prevents E-cadherin destabilization is also provided.

E-cadherin destabilization during cataract formation is associated with matrix metalloproteinases (MMP). The activity of MMPs can be blocked by MMP inhibitors (MMPIs). Thus, in a preferred embodiment of the invention, a therapeutically effective amount of a matrix metalloproteinase inhibitor (MMPI) is provided for the treatment or prevention of cataract. In another aspect of the invention, a device containing a MMPI is provided for the treatment or prevention of cataract. In a further aspect of the invention, a method for the treatment or prevention of cataract, comprising administering an effective amount of a MMPI is provided.

The methods, devices, and compositions of the present invention are useful for the treatment of various types of cataracts. For example, they may be useful in the treatment of secondary cataracts, which frequently occur following steroid treatment cataracts. Subcapsular cataracts, including anterior subcapsular cataracts, are particularly amenable to treatment according to the methods of the present invention.

The invention broadly relates to any agent that leads to a decrease in E-cadherin destabilization. This includes agents that directly inhibit activities of destabilizing agents as well as to agents that affect expression. According to the present invention, any agent that reduces the activity or expression of agents that cause Ecadherin destabilization can be used for the prevention or treatment of cataract. In a preferred embodiment an agent that inhibits MMP is provided for the treatment or prevention of cataract.

As used herein “inhibition of MMP” includes inhibition of MMP activity, as well as inhibition of MMP production regardless of the mechanism of activity or production. This inhibition can be caused directly, e.g. by binding to MMP or its binding partner, by MMP inhibitors or MMP antibodies or by preventing it acting as a proteinase. The inhibition can also be caused indirectly, for example by inhibiting a pathway that results in MMP production. Inhibition causes a reduction in the MMP activity regardless of the exact mechanism of inhibition.

As used herein an “MMP inhibitors” is an agent that directly or indirectly inhibits MMP activity. This includes an agent that blocks MMP activity or an agent that blocks a pathway of MMP production. The agent causes a reduction in MMP activity in the eye regardless of the mechanism of its action. The agent can also be a nucleic acid encoding the inhibitory agent such as a cDNA or genomic DNA. It could also be an RNA or DNA encoding MMP inhibitory activity such as an MMP antisense RNA or DNA.

Preferred matrix metalloproteinase inhibitors for use in the present invention include, but are not limited to, ((2R)-[(4-Biphenylylsulfonyl)amino]-N-hydroxyl-3-phenylpropionamide); actinonin (3-[[1-[[2-(hydroxymethyl)-1-pyrrolidinyl]carbamoyl]octano-hydroxamic acid); bromocyclic-adenosine monophosphate; N-chlorotaurine; BATIMISTAT (BB-94); CT1166 (N1 {N-[2-(morpholinosulphonylamino)-ethyl]-3-cyclohexyl-2-(S)propanamidyl}-N4-hydroxy-2-(R)-[3-(4-methylphenyl)propyl]-succinamide); estramustine (estradiol-3-bis(2-chloroethyl)carbamat-e); eicosa-pentaenoic acid; MARIMASTAT (BB-2516); matlystatin-B; peptidyl hydroxamic acids; N-phosphonalkyl dipeptides; protocatechuic aldehyde (3,4-dihydroxybenzaldehyde); Ro-31-7467 (2-[(5-bromo-2,3-dihydro-6-hydroxy-1,3-dioxo-1H-benz[de]isoquinol-in-2-yl)methyl](hydroxy)-[phosphinyl]-N-(2-oxo-3-azacyclotridecanyl)-4-met-hylvaleramide); tetracyclines; 1,10-phenanthroline (o-phenanthroline[4-(N-hydroxyamino)-2R-isobutyl-3S-(thiopen-2-ylthiomethyl)-succinyl]-L-p-henylalanine-N-methylamidecarboxyalkylamino-based compounds; chelators (EDTA, cysteine, acetylcysteine, D-penicillamine, and gold salts); bis(dioxopiperzaine); NEOVASTAT; KB-R7785; ILOMASTAT; RPR-122818; SOLIMASTAT; BB-1101; BB-2983; BB-3644; SMS-275291; D-1927, D-5410; CH-5902, CH-138; CMT-3; DERMOSTAT; DAC-MMPI; RS-1130830 and RS-113-080; GM-1339; GI-155704A; ONO-4817; AG-3433, AG-3088, PRINOMASTAT; CP-544439; POL-641: SC-964; 30-2590; PNU-142769; SU-5402; PGE-2946979, PGE4304887; fibrolase-conjugate; EF-13; S-3304; CGS-25015 and CGS-27023A; XR-168; RO1130830, D-9120; BB-2827; BB-1101 (2S-allyl-N1-hydroxy-3R-isobutyl-N4-(1S-methylcarbamoyl-2-phenylethyl)-su-ccinamide), BB-2983, solimastat (N′-[2,2-Dimethyl-1(S)-[N-(2-pyridyl)carba-moyl]propyl]-N4-hydroxy-2(R)-isobutyl-3(S)-methoxysuccinamide), N4-hydroxy-N1-[2-(methylamino)-2-oxo-1-(phenylmethyl)ethyl]-2-(2-methylpr-opyl)-3-[(2-thienylthio)methyl]-, [2R-[1(S*),2R*,3 S*]]-[CAS]), rebimastat (L-Valinamide, N—((2S)-2-mercapto-1-oxo-4-(3,4,4-trimethyl-2,5-dioxo-1-imidazolidinyl)butyl)-L-leucyl-N,3-dimethyl-[CAS]); PS-508; CH-715; nimesulide (Methanesulfonamide, N-(4-nitro-2-phenoxyphenyl)-[CAS]-), hexahydro-2-[2(R)-[1 (RS)-(hydroxycarbamoyl)-4-phenylbutyl]nonanoyl]-N—(-2,2,6,6-etramethyl-4-piperidinyl)-3(S)-pyridazine carboxamide, Cipemastat (1-Piperidinebutanamide, .beta.-(cyclopentylmethyl)-N-hydroxy-Gamma-oxo-A-lpha-[(3,4,4-trimethyl-2,5-dioxo-1-imidazolidinyl)methyl]-,(AlphaR,.beta.R-)-[CAS]), 5-(4′-biphenyl)-5-[N-(4-nitrophenyl)piperazinyl]barbituric acid, 6-methoxy-1,2,3,4-tetrahydro-norharman-1-carboxylic acid, Ro-31-4724 (L-Alanine, N-[2-[2-(hydroxyamino)-2-oxoethyl]-4-methyl-1-oxopentyl]-L-le-ucyl-, ethyl ester[CAS]), N-hydroxy-2,2-dimethyl-4-((4-(4-pyridinyloxy)phe-nyl)sulfonyl)-, (3R)-[CAS]), PNU-142769 (2H-isoindole-2-butanamide, 1,3-dihydro-N-hydroxy-Alpha-[(3S)-3-(2-methylpropyl)-2-oxo-1-(2-phenyleth-yl)-3-pyrrolidinyl]-1,3-dioxo-, (AlphaR)-[CAS]), (S)-1-[2-[[[(4,5-Dihydro-5-thioxo-1,3,4-thiadiazol-2-yl)amino]-carbonyl]amino]-1-oxo-3-pentafluoro-phenyl)propyl]-4-(2-pyridinyl)piperazine, SC77964, PNU-171829, N-hydroxy-2(R)-[(4-methoxybenzene-sulfonyl)(4 picolyl)amino]-2-(2-tetrahy-drofuranyl)-acetamide, L-758354 ((1,1′-Biphenyl)-4-hexanoic acid, Alpha-butyl-Gamma-(((2,2-dimethyl-1-((methylamino)carbonyl)propyl)amino)c-arbonyl)-4′-fluoro-, (AlphaS-(AlphaR*,GammaS*(R*)))-[CAS]); antibodies; and analogues or derivatives thereof. A more extensive list of examples of MMPs can be found in United States Patent Application No. 2004/0192658, as well as the patents cited therein, which are hereby incorporated by reference.

Broad-spectrum inhibitors that inhibit more than one type of MMP are preferred in one aspect of the Invention. One such example is GM6001. Also preferred are inhibitors that are capable of inhibiting MMP2, MMP9 or both MMP2 and MMP9. All of the various inhibitors can be used individually or in combination.

The MMPI of the present invention is preferably provided as pharmaceutical compositions that are suitable for application to the eye. The dosage of the compositions may vary depending on the route of administration and the severity of the disease. The dosage may also be adjusted depending on the body weight, age, sex, and/or degree of symptoms of each patent to be treated.

The inhibitors of the present invention are provided in a therapeutically effective amount that provides adequate inhibition without toxicity. Preferred concentrations of the inhibitor range from about 1 to 500 μg/ml, preferably 1 to 250 μg/ml, more preferably 1 to 100 μg/ml and most preferably 5 to 25 μg/ml. The inhibitor may be provided in combination with other pharmaceutically active agents. For example, other enzyme inhibitors or cytokine inhibitors may be co-administered with the MMPI. The frequency of administration depends on the formulation. For example, it may be desirable to apply eye drops at least once per day, preferably 2 to 5 times per day.

In a preferred embodiment, the composition comprises eye drops, injectable solutions or eye ointments. These pharmaceutical compositions can be formulated by admixing, diluting or dissolving the MMPI, optionally, with appropriate pharmaceutical additives such as excipients, disintegrators, binders, lubricants, diluents, buffers, isotonicities, antiseptics, moistening agents, emulsifiers, dispersing agents, stabilizing agents and dissolving aids in accordance with conventional methods and formulating in a conventional manner depending upon the dosage form. For example, eye drops can be formulated by dissolving an MMPI in sterilized water in which a surface active agent is dissolved and optionally adding appropriate pharmaceutical additives such as a preservative, a stabilizing agent, a buffer, an isotonicity, an antioxidant and a viscosity improver.

Injectable solutions can be directly injected into the cornea, crystalline lens and vitreous or their adjacent tissues using a fine needle. A solution can be also used as an intraocular perfusate.

The compositions of the present invention can be administered as sustained release preparations. For example, an MMPI can be incorporated into a pellet or microcapsule of a sustained release polymer as a carrier, and the pellet or microcapsule surgically in planted into the tissues to be treated. An intraocular lens that incorporates an MMPI composition can also be used to deliver the medicament.

One preferred mode for delivery of a composition of the invention to the eye is via a contact lens. The lens may be provided already treated with the inhibitor composition. Alternatively, the components for preparing a coated lens are provided as lyophilized powders for reconstitution, as concentrated solutions, or ready for use. The compositions can be provided as kits for single or multi-use. The kits may include disposable contact lens and the inhibitor composition. The composition is provided in a container, either as a concentrate that is diluted prior to use in an appropriate diluent for use in the eye or at the ready-to-use concentration. Preferably, single dosages are provided in sterile vials.

The compositions of the present invention can also be provided for administration via any route, including i.v, i.p. i.m., s.c, etc. In one preferred embodiment they are provided in a formulation for oral or nasal administration. The composition is administrated to prevent and/or treat cataract associated with MMP activity.

The compositions can be provided in various types of formulation, such as tablets, granules, capsules and injectables by using conventional methods. Such formulations may contain vehicles such as binders, disintegrators, thickeners, emulsifiers, resorptioners, corrigents, buffering agents, surfactants, solubilizing agents, preservatives, suspending agents, isotonicities, stabilizers, and pH adjusting agents.

The present invention provides for a pharmaceutical composition or medicament containing an agent that prevents or reduces E-adherin destabilization. While this description has focused on MMPIs as agents that prevent E-cadherin destabilization, it is clearly apparent that other agents which inhibit E-adherin destabilization could also be used according to the present invention since the inventors are the first to identify the role of E-cadherin destabilization in cataract formation. It is also apparent that the use of MMPI as a treatment for cataract is not limited to their effects on E-cadherin destabilization. MMPI may have other effects that help prevent or reduce cataract that have not yet been identified. The compositions of the invention are not limited by their mechanism of action. Exemplary modes of practicing the invention and its utility are demonstrated in the examples at the end of this disclosure.

The above disclosure generally describes the present invention. It is believed that one of ordinary skill in the art can, using the preceding description, make and use the compositions and practice the methods of the present invention. A more complete understanding can be obtained by reference to the following specific examples and the Figures. These examples are described solely to illustrate preferred embodiments of the present invention and its utility and are not intended to limit the scope of the invention. Changes in form and substitution of equivalents are contemplated as circumstances may suggest or render expedient. Other generic configurations will be apparent to one skilled in the art. All journal articles and other documents such as patents or patent applications referred to herein are hereby incorporated by reference.

### EXAMPLES

Although specific terms have been used in these examples, such terms are intended in a descriptive sense and not for purposes of limitations Methods of biology, molecular biology, chemistry and physics referred to but not explicitly described in the disclosure and these examples are reported in the scientific literature and are well known to those skilled in the art.

### Example 1

### Ex-vivo Rat Lens Cataract Model

The previously established TGFβ-induced rat lens model was utilized for these studies. Briefly, lenses were obtained from adult male Wistar rats and cultured in 3.5 ml of serum free M199 medium supplemented with 50 IU/ml penicillin, 50 μg/ml streptomycin and 2.5 μg/ml fungizone (Amersham Biosciences) overnight. The following day lenses were either left untreated or treated with TGFβ2 (R&D Systems) at a final concentration of 1 or 2 ng/ml. Some lenses were co-treated with TGFβ2 and the MMP1, GM6001 (liomastat) (Chemicon International) at concentrations ranging from 10 to 25 μM or the MMP-2-9 inhibitor ((2R)-[(4-Biphenylylsulfonyl)amino]-N-hydroxyl-3-phenylpropionamide) (Chemicon) concentrations of either 10 and 25 μM. The GM6001 negative control (N-t-Butoxycarbonyl-L-leucyl-L-tryptophan Methylamide) (Calbiochem) (25 μM) was also employed. Lenses were then harvested at subsequent time-points of 2, 4 or 6 days and then photographed using a digital camera mounted to a dissecting scope. The lenses were then fixed for histology and immunofluorescence, or used for optical analysis. The conditioned media was also collected from each treatment group for zymography and western blot analysis.

### Example 2

### Optical Analysis

Lens optical qualifies (the average back vertex distance, BVD) and sharpness of focus (BVD error) were assessed using the automated laser scanning system that was developed at the University of Waterloo. This system consists of a single collimated scanning helium-neon laser source that projects a thin (0.05 mm) laser beam onto a plain mirror mounted at 45° on a carriage assembly. A digital camera captures the actual position and slope of the laser beam at each step. When all steps have been made, the captured data for each step position is used to calculate the back vertex distance for each position and the difference in that measurement between beams. Each lens studied was suspended within the chamber on a beveled washer, of inner diameter ranging from 3.0 mm. Back vertex focal length (spherical aberration) was measured for 20 beam positions across each lens. Back vertex distance (BVD, m) is defined as the measurement of the distance from the surface of the lens to the focal point where the laser beam crosses the optical axis of the lens being scanned. The instrument first locates the optical centre of the lens, the position of zero or minimal deviation of the beam. Back vertex distance is determined for a set number of beam positions on either side of the centre. Normally, changes in back vertex distance as a function of eccentricity from the centre indicate the spherical aberration of the lens. Change in this distance (BVD error) affect the sharpness of focus and are a result of spherical aberration, or morphological irregularities. BVD error (mm) was calculated as the standard error of the mean of BVDs measured for a single lens by the scanning laser.

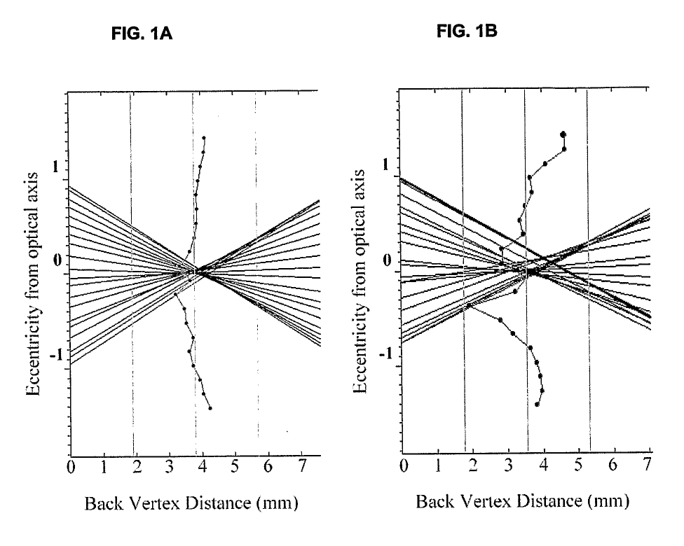

When portrayed graphically the average BVD for the lenses is plotted for each eccentric position. As shown in FIG. 1, the scatter plots represent the back vertex measurements (focal length) of a control lens (left) and a TGF-β treated lens (right). The Y-axis indicates the eccentricity of the laser beam from the optical axis and the X-axis demonstrates back vertex distance (BVD) measurements. Each point on the scatter plot represents the back vertex distance from each beam location. In a control (non-cataract us) lens there is little difference in back vertex distance demonstrating sharpness in focus. With less spherical aberration, the data points line up as a straight line. The poorer the quality of the lens, the greater the variation in BVD (BVD error) is with eccentricity, as shown for lenses treated with TGFβ. Since BVD error is a more sensitive measure of lens damage, the results are expressed in terms of BVD error.

### Example 3

### Effects of Various Treatments on Back Vertex Variability

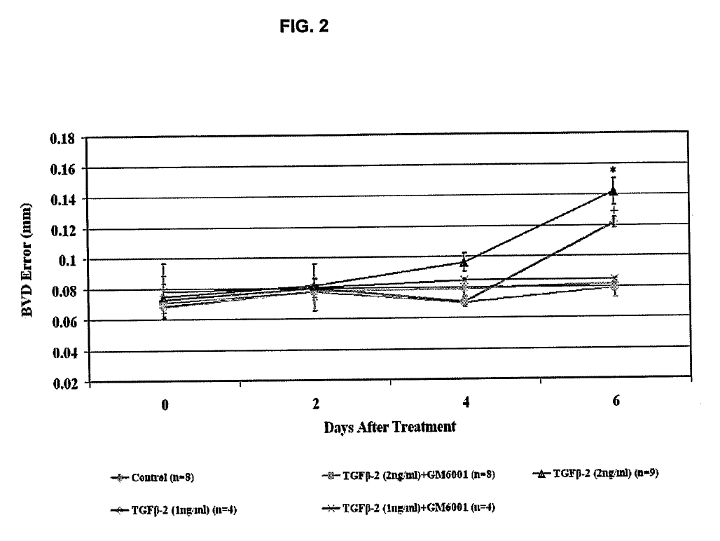

Repeated-measures analysis of variance (repeated-measures ANOVA) (SPSS™ 11.0 statistical software) was used to assess treatment, concentration of TGFβ, and temporal effects on the back vertex variability. The results of one experiment are shown in FIG. 2. This is a two-factor experiment with repeated measures on one factor: one within factor of lenses (time of optical scans) and one between factor of lenses (treatment group). The graph of FIG. 2 shows the change in back vertex variability (BVI) from day 0 (initial measurements before treatment) and 2, 4 and 6 days after treatment. Two concentrations of TGF-13 were used in this experiment. Analyses showed that there was both a treatment and a temporal effect. At day 6, both the TGF-β treated groups of lenses exhibited a significantly larger BVD error than the control group or the GM6001 co-treated lenses.

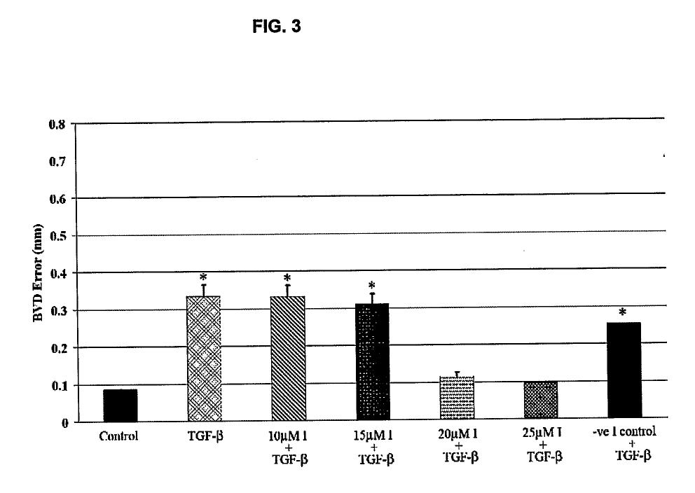

In a further experiment, lenses were untreated or treated with TGF-β plus four different concentrations of GM6001 for six days. The results are shown in FIG. 3. One way analysis of variance was used for analyzing data in FIG. 3, which examines the dosage effect of GM6001 on TGFβ-induced BVD error. A probability value (p-value)≦s 0.05, indicating a 95% confidence interval, was considered to be significant. Treatment with TGFβ for 6 days resulted in BVD errors that were statistically greater than in controls.

A dose dependent effect of the GM6001 inhibitor in preventing the TGFβ-induced cataracts was also observed as seen in FIG. 3. This bar graph represents the back vertex variability (BVD error, m) of lenses left untreated, or treated with TGFβ (2 ng/ml), or TGFβ (2 ng/ml) plus 4 different concentrations of GM6001 (I)(10 μM, 15 μM, 20 μM and 25 μM) for 6 days. These measurements show a decrease in BVD error as the GM6001 concentration increases to 25 μM. BVD errors from TGFβ treated lenses co-treated with 25 μM GM6001 and 20 μM GM6001 were not significantly different from the control lenses. In contrast, lenses treated with TGFβ (2 ng/ml) alone, or with TGFβ (2 ng/ml) and GM6001 at 10 and 15 μM had BVD errors that were significantly different from control lenses. In addition, lenses treated with TGFβ and the negative control for GM6001 (25 μM) had BVD errors that were also significantly different from the controls. These results demonstrate the dramatic effect that MMPI can have on the development of cataracts.

### Example 4

### Histology and Immunofluorescence

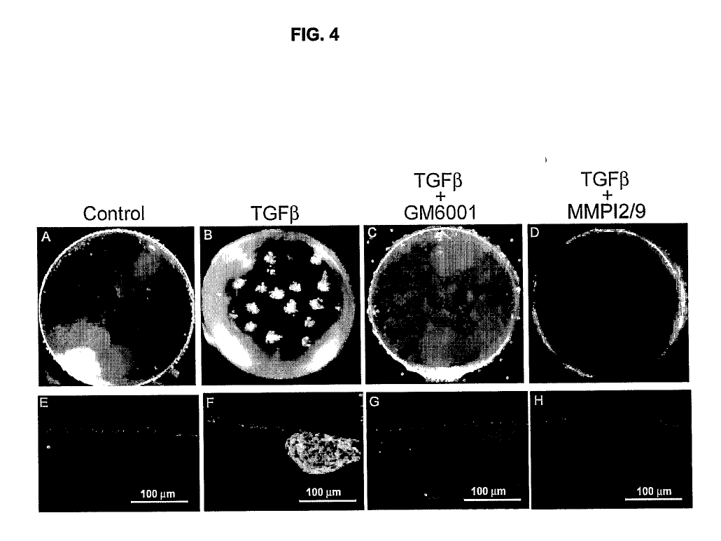

Lenses were collected from different treatment groups and fixed overnight in 1:99 acetic acid: ethanol solution, dehydrated, embedded in paraffin, and processed for routine histology. For histological analysis, 5 μm sections were stained with hematoxylin and eosin. Immunofluorescence was performed on 5 μm thick paraffin-embedded sections. Sections were incubated with primary antibody specific for alpha smooth muscle actin (α-SMA, 1:100, Sigma) and bound primary antibodies were visualized with a fluorescein-isothiocyanate (FITC) anti-mouse secondary antibody, (1:50, Jackson ImmunoResearch Laboratories). All sections were mounted in Vectasheild mounting medium with 4′,6-Diaminodino-2-Phenylindol (DAPI, Vector Laboratories) to visualize the nuclei. The results are shown in FIG. 4.

The MMP inhibitor GM₆₀₀₁ was employed in the rat subcapsular cataract model in order to determine whether it could effectively suppress TGFβ-induced subcapsular cataract formation. To carry out these experiments, excised rat lenses were treated with exogenous TGFβ for a period of 6 days. The results are shown in FIG. 4. An untreated control lens (A), a lens treated with TGF-β (2 ng/mL) (B), a lens co-cultured with TGFβ (2 ng/mL) and GM6001 (25 μM) (C) and a lens co-cultured with TGF-β and MMPI-2/9 (2 umol/L) (D) are shown following 6 days of culture. The TGFβ (2 ng/ml) treated lens (B) exhibited distinct subcapsular plaques unlike the untreated lens (A) or the lens co-cultured with GM6001 (25 μM) (C) or MMPI (D) that remained devoid of opacities. Immunofluorescent localization of αSMA in cross-sections of lenses revealed strong immunoreactivity of αSMA (green) in sections of lenses treated with TGFβ (2 ng/ml) (F), confirming the presence of subcapsular plaques, whereas control lenses (E) and lenses cocultured with TGFβ (2 ng/ml) and GM6001 (25 μM) (G) or lenses co-cultured with TGFβ (2 ng/ml) and MMP 2/9 Inhibitor (101M) (H) showed no observable immunoreactivity to αSMA, All sections were mounted in a medium with DAPI to co-localize the nuclei (blue). Scale bars represent 100 μm.

### Example 5

### Correlaton of Enhanced Secretion of MMP-2 and MMP-9 in TGFβ-Induced ASC Formation

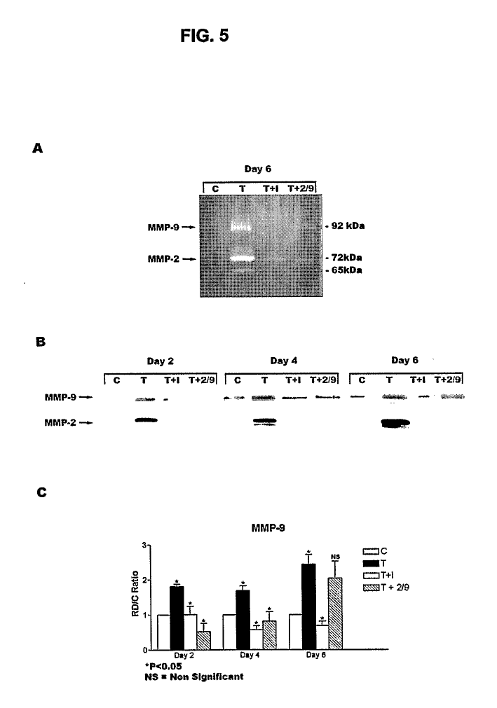

Conditioned media from all treatment groups was concentrated using 3.5 ml 10 K Microsep concentrating devices (Viva Sciences), Prior to concentration, refrigerated media was warmed to 37° C. The media was centrifuged at 1000 g (at room temperature) for 5 min to pellet any debris prior to loading. Each device was loaded with equal volume of supernatant and the concentration was performed by centrifugation at 25° C. for 30 min at 4000 g. An equal volume of each concentrate was electrophoresed on 10% SDS-polyacrylamide gels containing either 1 mg/ml gelatin or 2% beta-casein at a final concentration of 0.1% as the substrate. Following electrophoresis the gels were developed as described previously and stained in 0.5% coomassie brilliant blue for 1 hr followed by destaining with 10% iso-propanol. Sites of gelatinase or caseinase activity were detected as clear bands against a background of uniform staining, which was digitally photographed. The results are shown in FIG. 5A.

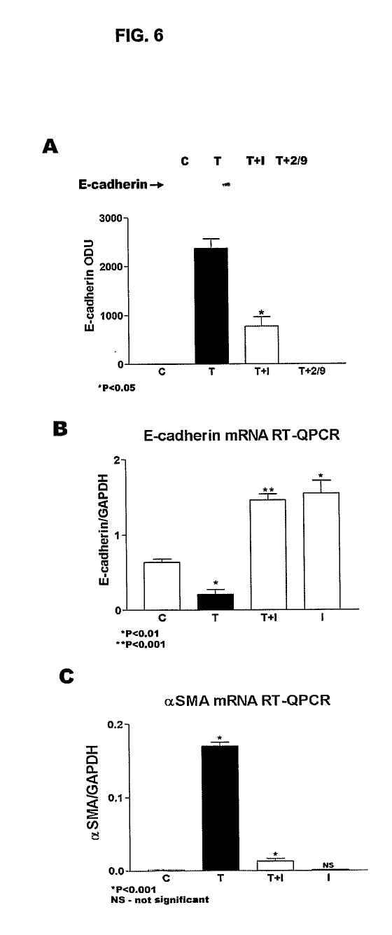

To examine the timing and level of induction of MMPs In subcapsular cataract formation zymography was performed on conditioned media of lenses from three different treatment groups (control, TGFβ, TGFβ plus GM6001 (25 μM)) and TGFβ plus MMPI-2/9 at the 6 day time point. The results are shown in FIG. 6A.

Conditioned media from all treatment groups showed no MMP activity on the casein gels indicating the absence of collagenolytic activity (data not shown). On the gelatin gels, conditioned media from all treatment groups exhibited distinct bands, indicating the presence of MMPs with gelatinolytic activity (FIG. 5A). Conditioned media from control lenses exhibited expression of a 92-kDa band, corresponding to the proform of MMP-9. In comparison, conditioned media from lenses treated with TGFβ exhibited the MMP-9 band, which appeared elevated relative to the control lens media, as well as additional bands at 62, 65 and 72 kDa corresponding to active and proforms of MMP-2. Media obtained from lenses co-treated with TGFβ and GM6001 or MMPI-2/9 at the 6 day time point exhibited reduced levels of all gelationolytic bands relative to that of TGFβ treated lenses.

To confirm the identity of the specific MMPs corresponding to the gelatinolytic bands in the zymograms and quantitate their levels over the 6 day time period, concentrated conditioned media from the treatment groups, control, TGFβ, TGFβ plus GM6001 (25 μM) and TGFβ plus MMPI-2/9, from three treatment times days 2, 4 and B, was subjected to western blot analysis using antibodies specific for both MMP-2 and MMP-9. Equal volumes of sample were electrophoresed on a 10% SDS-polyacrylamide gel. The resolved bands were electro-transferred onto a nitrocellulose membrane (Pall Corporation). Membranes were blocked with 5% skimmed milk powder in Tris-buffered saline (50 mM Tris base, NaCl pH 8.5) and 0.1% Tween-20 and then incubated overnight at 4° C. with a polyclonal antibody generated against MMP-9 (1:500; Chemicon International) or MMP-2 (1:500:Chemicon International) or E-adherin (1:1500; ED Transduction Laboratories).

Following this incubation, membranes were probed with an HRP-conjugated secondary antibody (1:7500;Amersham Biosciences) and ECL detection reagents (Amersham Biosciences). The western blots were scanned by a densitometer and analyzed by image quantification software (ImageJ, NIH, USA). The relative density versus control ratio (RD/C) was estimated using Graph Pad Prism Program (GraphPad). Quantitative data were analyzed statistically using a student's t-test and expressed as ± standard deviation. A value of p<0.05 was considered significant. The results are shown in FIG. 5B.

Blots developed with an MMP2 specific antibody revealed the presence of latent and active species of MMP2 in conditioned media from lenses treated with TGFβ, whereas the control lenses did not exhibit detectable levels of MMP-2 protein as shown in FIG. 58. Conditioned media from the lenses co-cultured with TGFβ and GM6001 or MMPI-2/9 showed undetectable levels, similar to controls. The blots probed with the MMP-9 specific antibody revealed the presence of a band at the predicted molecular size of 92 kDa, for the proform of MMP9. The results are shown in FIG. 5B.

The western blot data for MMP-9 from three separate experiments were analyzed by densitometry and the results are shown in FIG. 5C. Values are expressed as the Relative Density versus control ratio (RD/C)± standard deviation of 3 blots. Note a significant upregulation (*P<0.001) of MMP-9 in the conditioned media of TGFβ (2 ng/ml) (T) treated lenses as compared to control. A significant reduction (**P<0.001) was observed in the expression of MMP-9 in conditioned media of lenses co-treated with TGFβ (2 ng/ml) and GM6001 (25 μM) (T+I) as compared to TGFβ (2 ng/ml) (T).

Similar to the zymography results, constitutive MMP-9 protein expression was evident in the conditioned media of control lenses. In comparison, media from lenses treated with TGFβ exhibited significantly higher levels of MMP-9, following 4 and 6 days of treatment (FIG. 5B,C). Media from lenses co-treated with TGFβ and GM6001 showed levels of MMP-9 that were significantly attenuated relative to those treated with TGFβ alone. Cob treatment with MMPI-2/9 also showed a significant attenuation of MMP-9. Lenses treated with either MMPI alone exhibited levels of MMP-2 and MMP-9 similar to that of controls.

### Example 6

### TGFβ Induced E-Cadherin Shedding

To determine whether TGFβ treatment of the rat lens results in an induction of E-cadherin shedding and whether this can be modulated by MMPIs, previously concentrated conditioned media was obtained from the 6 day treatment groups outlined earlier, and subjected to western blot analyses using an antibody specific for extracellular domain of E-cadherin, and used in previous studies to detect the presence of soluble E-cadherin fragments. The results are shown in FIG. 6A. The western blot revealed the presence of an approximately 72 kDa E-cadherin fragment in the conditioned media from lenses treated with TGFβ (2 ng/ml) (T) that was not detected in media from control lenses (C). Suppression in the levels of the E-adherin fragment was observed in media from lenses co-cultured with TGFβ and GM6001 (25 μM) (T+I) In the group treated TGFβ and MMPI-2/9, the E-adherin fragment was undetectable. Lenses treated with either inhibitor alone exhibited undetectable levels of E-cadherin. The data were confirmed by densiometric analysis of immunoblots for E-cadherin.

E-cadherin mRNA expression in the rat lens epithelial region was determined for lenses from different treatment groups, TGF-β, TGF-β plus GM6001 (25 μM), TGF-β plus MMPI-2/9 or control (6 day treatment time), using Real time quantitative PCR, (RT-QPCR). For these experiments, cryostat sections of lenses were subjected to Laser Capture Microdissection (LCM) in order to specifically isolate the cells. LCM was used to further ensure that cells of the subcapsular plaques were obtained and not left adherent to the underlying fiber cell mass, which may have occurred with manual dissection. Following treatment, lenses were placed in a cryostat mould containing Tissue-Tek OCT (Sakura Finetek Torrance, Calif.), and frozen on dry ice then stored at −70° C. The frozen tissue was then sectioned at 5-8 μm in a cryostat, mounted on non-coated clean glass slides, and stored again at −70° C. Immediately before Laser Capture Microdissection (LCM), the frozen sections were thawed for 10 seconds and then stained with HistoGene™LCM Frozen Section Staining kit (Arcturus, Mountain View, Calif.) using the protocol provided with strict adherence to RNAse-free conditions. The slides were then dried for 5 minutes after which LCM of the tissue was completed within two hours. LCM was then performed using the PixCell II (Arcturus), as described by others. The HistoGene stain allowed for the identification of the general morphology of the epithelium. Cells from the epithelium of the lens were then captured on CapSure Macro LCM Caps (Arcturus) using the PixCell II LCM Microscope (Arcturus) with a minimal beam diameter of 7.5 μrm. Total cellular RNA was then extracted from lifted cells using a PicoPure™ RNA Isolation kit (Arcturus Engineering Inc). Purified RNA was analyzed both qualitatively and quantitatively using an Agilent 2100 Bioanalyser (Agilent Technologies, Foster City, Calif., USA). Standard 20 μl reverse transcription reactions were preformed (SuperScript II, Life Technologies). The quality of the recovered cDNA was measured using a microfluidic gel analyzer (Agilent Technologies). E-Cadherin gene expression from recovered cDNA was analyzed using RT-PCR using a 96 well TaqMan optical reaction plate format on an ABI Prism 7700 sequence detection system (Applied Biosystems, Foster City, Calif., USA). RNA was normalized to 18S for each reaction. Each 25 μl PCR reaction (including controls) contained TaqMan Universal master mix, gene-specific forward and reverse primers (Mobix, Hamilton), and probes for target and endogenous control genes (Applied Biosystems). Serial dilutions (1-5 fold) of standard samples were set up in separate wells in duplicate, for both 18S and E-cadherin gene targets. Standard and unknown samples were added in a volume of 5 μl. Thermal cycling parameters consisted of the following: 2 minutes at 50° C., 10 minutes at 95° C. followed by 40 cycles of 15 seconds at 95° C. and 1 minute at 60° C. The number of target gene copies was calculated from a standard curve generated in parallel with each batch of samples. A linear relationship was detected over at least five orders of magnitude. In all experiments, the correlation coefficient was between 0.997 and 0.986. The normalization of samples was performed by dividing the number of copies of E-Cadherin by the number of copies of 18S. All PCR for E-cadherin cDNA quantification were performed using standard cDNA dilution curves. Quantitative data were analyzed statistically using a student's t-test and expressed as ±standard deviation. A value of p<0.05 was considered significant.

The results are shown in FIG. 6B. RT-QPCR findings revealed that while E-cadherin mRNA was detected in the normal lens epithelium, it was suppressed nearly 4-fold in the plaque tissue of TGFβ treated lenses. In comparison, E-cadherin levels in lens epithelium of lenses co-treated with TGFβ and GM6001 or MMPI-2/9 were significantly higher than those treated with TGFβ alone and also significantly higher than that of controls. These findings suggest that GM6001 not only prevented the attenuation of E-cadherin mRNA expression induced by TGFβ but may have further stabilized constitutive E-adherin mRNA levels in the rat lens epithelium.

For comparison, levels of α-SMA mRNA were examined in the same tissues. The results are shown in FIG. 6C. There was little or no expression of α-SMA mRNA in the cells obtained from the epithelial region of control lenses whereas expression was significantly induced in the plaque tissue of TGFβ-treated lenses. α-SMA mRNA levels in the LECs co-treated with the MMPIs were substantially reduced compared to the plaque cells of TGFβ-treated lenses.

### Example 10

### Role of MMP-9 in Cataract Development

Matrix Metalloproteinases (MMPs) have been shown to play a functional role in epithelial to mesenchymal transition (EMT) during TGFβ-induced anterior subcapsular cataract (ASC) formation. A new model of ASC using adenoviral gene delivery (AdTGFβ1) was employed in MMP-9 knock out (KO) mice to examine the requirement of MMP-9 expression in TGFβ-induced cataract formation.

Wild type and MMP-9 KO mice, on an FVB background, aged 6-8 weeks were injected with recombination-deficient adenovirus containing cDNA coding for active porcine TFGβ1 (AdTGFβ1) or control vector (AdGFP) containing green fluorescent protein, into the anterior chamber of the eye. The animals were sacrificed at 4 and 21 days post-injection. The eyes were dissected, fixed for histology and stained with Masson's Trichrome or used for immunohistochemical localization of a smooth muscle actin (α-SMA).

In the wild-type mice (day 4 (n=7); day 21 (n=2)) post-injection, adenivirally delivered active TGFβ1, resulted in the formation of distinct anterior subcapsular plaques that were immunoreactive to α-SMA demonstrative of EMT. Additionally, Masson's trichrome stain revealed aberrant matrix deposition in the fibrotic plaques of the wild-type mice at day
21. In contrast to these findings, MMP9 KO mice at 4 days (n=5) and 21 days (n=6) post-injection did not exhibit plaques and the lens epithelial cells showed no reactivity to α-SMA. In addition, no aberrantly deposited matrix was observed in the MMP9 KO lenses. The eyes injected with the control vector, AdGFP (day 21), did not show any of the cataractous changes.

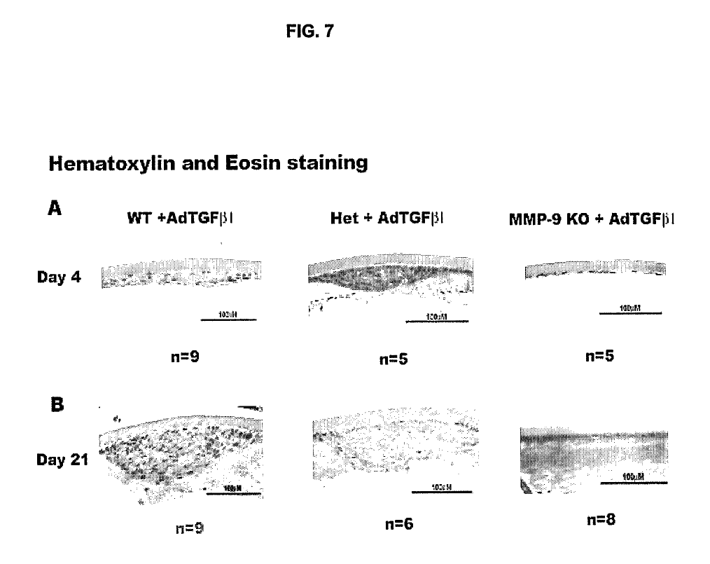

FIG. 7 illustrates hematoxylin and Eosin staining in cross-sections of Wild-type (WT), Heterozygous (Het), and MMP-9 KO mouse eyes treated with AdTGFβ1 Four (A) and 21 (B) days post-injection, the MMP-9 wild-type and heterozygous mouse eyes treated with AdTGFβ1 showed zones of focal multi-layering of epithelial cells (plaques) beneath the intact anterior lens capsule. In contrast to these findings, MMP-9 KO mice at 4 (A) and 21 (B) days post-injection of AdTGFβ1 did not exhibit any subcapsular plaques or multilayering of lens epithelial cells.

A list of references is appended and all citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

1. McCarty C A, Taylor H R: Recent developments in vision research: light damage in cataract. Invest Opthalmol Vis Sci 1996, 37:1720-1723
2. Griep A E, Zhang P: Lens Cell Proliferation. Development of the Ocular Lens. Edited by Lovicu F J, Robinson M L. New York, Cambridge University Press, 2004, pp 191-213
3. Lang R A, McAvoy J W: Growth Factors in Lens Development. Development of the Ocular Lens. Edited by Lovicu F J, Robinson M L. New York, Cambridge University Press, 2004, pp 261-289
4. Sasaki K, Kojima M, Nakaizumi H, Kitagawa K, Yamada Y, Ishizaki H: Early lens changes seen in patients with atopic dermatitis applying image analysis processing of Scheimpflug and specular microscopic images. Opthalmologica 1998, 212:8894
5. Font R, S A B: A light and electron microscopic study of anterior subcapsular cataracts. American Journal of Opthalmology 1974, 78:972-984
6. Novotny G E, Pau H: Myofibroblast-like cells in human anterior capsular cataract. Virchows Arch A Pathol Anat Histopathol 1984, 404:393-401
7. Hay E D: An overview of epithelio-mesenchymal transformation. Acta Anat (Base1) 1995, 154:8-20
8. Lovicu F J, Schulz M W, Hales A M, Vincent L N, Overbeek P A, Chamberlain C G, McAvoy J W: TGFbeta induces morphological and molecular changes similar to human anterior subcapsular cataract. Br Opthalmol 2002, 88:220-226
9. Kappelhof J P, Vrensen G F: The pathology of after-cataract. A minireview. Acta Opthalmol Suppl 1992:13-24
10. Marcantonio J M, Syam P P, Liu C S, Duncan G: Epithelial transdifferentiation and cataract in the human lens. Exp Eye Res 2003, 77:339-346
11. Alexander C M, Werb Z: Proteinases and extracellular matrix remodeling. Curr Opin Cell Biol 1989, 1:974-982
12. Sivak J M, Fini M E: MMPs in the eye: emerging roles for matrix metalloproteinases in ocular physiology. Prog Retin Eye Res 2002, 21:1-14
13. Snine A, Plantner J J: Membrane type-1 matrix metalloproteinase in human ocular tissues. Curr Eye Res 1997, 16:925-929
14. Reponen P, Sahlberg C, Munaut C. Thesleff I, Tryggvason K: High expression of 92-kD type IV collagenase (gelatinase B) in the osteoclast lineage during mouse development. J Cell Biol 1994, 124:1091-1102
15. Mohan R, Rinehart W B, Bargagna-Mohan P, Fini M E: Gelatinase B/lacZ transgenic mice, a model for mapping gelatinase B expression during developmental and injury-related tissue remodeling. J Biol Chem 1998, 273:25903-25914
16. Seomun Y, Kim J, Lee E H, Joo C K: Overexpression of matrix metalloproteinase-2 mediates phenotypic transformation of lens epithelial cells. Biochem J 2001, 358:41-48
17. Richiert D M, Ireland M E: Matrix metalloproteinase secretion is stimulated by TGF-beta in cultured lens epithelial cells. Curr Eye Res 1999, 19:269-275
18. Wormstone I M, Tamiya S, Anderson I, Duncan G: TGF-beta2-induced matrix modification and cell transdifferentiation in the human lens capsular bag. Invest Opthalmol Vis Sci 2002, 43:2301-2308
19. Tamiya S. Wormstone 1M, Marcantonio J M, Gavrilovic J, Duncan G: Induction of matrix metalloproteinases 2 and 9 following stress to the lens. Exp Eye Res 2000, 71:591-597
20. Kawashima Y, Saika S, Miyamoto T, Yamanaka O, Okada Y, Tanaka S, Ohnishi Y: Matrix metalloproteinases and tissue inhibitors of metalloproteinases of fibrous humans lens capsules with intraocular lenses. Curr Eye Res 2000, 21:962-967
21. Stemlicht M D, Lochter A, Syrnpson C J, Huey B. Rougier J P, Gray J W, Pinkel D, Bissell M J, Werb Z: The stromal proteinase MMP3/stromelysin-1 promotes mammary carcinogenesis. Cell 1999, 98; 137-146
22. George S J, Dwivedi A: MMPS, cadherins, and cell proliferation. Trends Cardiovasc Med 2004, 14:100-105
23. Ho A T, Voura E B, Soloway P D, Watson K L, Khokha R: MMP inhibitors augment fibroblast adhesion through stabilization of focal adhesion contacts and up-regulation of cadherin function J Biol Chem 2001, 276:40215-40224
24. Mei J M, Borhert G L, Donald S P, Phang J M; Matrix metalloproteinase(s) mediate(s) NO-induced dissociation of beta-catenin from membrane bound E-cadherin and formation of nuclear beta-catenin/LEF-1 complex. Carcinogenesis 2002, 23:2119-2122
25. Noe V, Fingleton 8, Jacobs K, Crawford H C, Vermeulen S, Steelant W, Bruyneel E, Matrisian L M, Mareel M: Release of an invasion promoter E-cadherin fragment by matrilysin and stromelysin-1. J Cell Sci 2001, 14:111-118
26, Hirai Y, Migita K, Honda S, Ueki Y, Yamasaki S, Urayama S, Kamachi M, Kawakami A, Ida H, Kita M, Fukuda T, Shibatomi K, Kawabe Y, Aoyagi T, Eguchi K: Effects of nitric oxide on matrix metalloproteinase-2 production by rheumatoid synovial cells. Life Sci 2001, 68:913-920
27. Hales A M, Chamberlain G O, McAvoy J W: Cataract induction in lenses cultured with transforming growth factor-beta. Invest Opthalmol V is Sci 1995, 36:1709-1713
28. Priolo S, Sivak J G, Kuszak J R, Irving E L: Effects of experimentally induced ametropia on the morphology and optical quality of the avian crystalline lens. Invest Opthalmol V is Sci 2000, 41:3516-3522
29. Bantseev V, McCanna D, Banh A. Wong W W, Moran K L, Dixon D G, Trevithick J R, Sivak J G: Mechanisms of ocular toxicity using the in vitro bovine lens and sodium dodecyl sulfate as a chemical model. Toxicol Sci 2003, 73:98-107
30. Oriowo O M, Cullen A P, Chou B R, Sivak J G: Action spectrum and recovery for in vitro UV-induced cataract using whole lenses. Invest Opthalmol V is Sci 2001, 42:2596-2602
31. Heussen C, Dowdle E B; Electrophoretic analysis of plasminogen activators in polyacrylamide gels containing sodium dodecyl sulfate and copolymerized substrates. Anal Biochem 1980, 102:196-202
32. Emmert-Buck M R, Bonner R F, Smith P D, Chuaqui R F, Zhuang Z, Goldstein S R, Weiss R A, Liotta L A: Laser capture microdissection. Science 1996, 274:998-1001
33. Bonner R F, Emmert-Buck M, Cole K, Pohida T, Chuaqui R, Goldstein S, Liotta L A: Laser capture microdissection: molecular analysis of tissue. Science 1997, 278:1481,1483
34. Lovicu F J, Steven P, Saika S, McAvoy J W: Aberrant lens fiber differentiation in anterior subcapsular cataract formation: a process dependent on reduced levels of Pax6. Invest Ophthalmol Vis Sci 2004, 45:1946-1953
35. John M, Jaworski C, Chen Z. Subramanian S, Ma W, Sun F, Li D, Spector A, Carper D: Matrix metalloproteinases are down-regulated in rat lenses exposed to oxidative stress. Exp Eye Res 2004, 79:839-846
36. Song W, Jackson K, McGuire P G: Degradation of type IV collagen by matrix metalloproteinases is an important step in the epithelial-mesenchymal transformation of the endocardial cushions. Dev Biol
37. Boyer A S, Erickson C P, Runyan R B: Epithelial-mesenchymal transformation in the embryonic heart is mediated through distinct pertussis toxin-sensitive and TGFbeta signal transduction mechanisms. Dev Dyn 1999, 214:81-91
38. Brown C B, Boyer A S, Runyan R B, Barnett J V: Requirement of type III TGF-beta receptor for endocardial cell transformation in the heart. Science 1999, 283:2080-2082
39. Steinhusen U, Weiske J. Badock V, Tauber R, Bommert K, Huber O: Cleavage and shedding of E-cadherin after induction of apoptosis. J Biol Chem 2001, 276:4972-4980
40. Lyu J, Kim J A, Chung S K, Kim K S, Joo C K: Alteration of cadherin in dexamethasone-induced cataract organcultured rat lens. Invest Opthalmol V is Sci 2003, 44:2034-2040
41. Nawrocki-Raby B, Gilles C, Polette M, Bruyneei I E, Laronze J Y, Bonnet N, Foidart J M, Mareel M, Birembaut P: Upregulation of MMPs by soluble E-cadherin in human lung tumor cells. Int J Cancer 2003, 105:790-795
42. Kerkviiet E H, Jansen I D, Schoenmaker T A, Docherty A J, Beertsen W, Everts V: Low molecular weight inhibitors of matrix metalloproteinases can enhance the expression of matrix metalloproteinase-2 (gelatinase A) without inhibiting its activation. Cancer 2003, 97:1582-1588
43. Lei T C, Vieira W D, Hearing V J: In vitro migration of melanoblasts requires matrix metalloproteinase-2: implications to vitiligo therapy by photochemotherapy. Pigment Cell Res 2002, 15:426-432

1. A method of treating or preventing cataract, said method comprising administering to a subject in need of such treatment, a therapeutically effective amount of an agent that inhibits E-cadherin destabilization.

2. A method according to claim 1 wherein cataract is a subcapsular cataract.

3. A method according to claim 2 wherein cataract is an anterior subcapsular cataract.

4. A method according to claim 1 wherein the agent is a matrix metalloproteinase inhibitor (MMPI).

5. A method according to claim 4 wherein the MMPI is a broad range MMPI.

6. A method according to claim 4 wherein the MMPI is selected from the group consisting of: ((2R)-[(4-Biphenylylsulfonyl)amino]-N-hydroxyl-3-phenylpropionamide); actinonin (3-[[1-[[2-(hydroxymethyl)-1-pyrrolidinyl]carbamoyl]-octano-hydroxamic acid); bromocyclic-adenosine monophosphate; N-chlorotaurine; BATIMISTAT (BB-94); CT1166 (N1 {N-[2-(morpholinosulphonylamino)-ethyl]-3-cyclohexyl-2-(S)-propanamidyl}-N4-hydroxy-2-(R)-[3-(4-methylphenyl)propyl]-succinamide); estramustine (estradiol-3-bis(2-chloroethyl)carbamat-e); eicosa-pentaenoic acid; MARIMASTAT (BB-2516); matlystatin-B; peptidyl hydroxamic acids; N-phosphonalkyl dipeptides; protocatechuic aldehyde (3,4-dihydroxybenzaldehyde); Ro-31-7467 (2-[(5-bromo-2,3-dihydro-6-hydroxy-1,3-dioxo-1H-benz[de]isoquinol-in-2-yl)methyl](hydroxy)-[phosphinyl]-N-(2-oxo-3-azacyclotridecanyl)-4-met-hylvaleramide); tetracyclines; 1,10-phenanthroline (o-phenanthroline[4-(N-hydroxyamino)-2R-isobutyl-3S-(thiopen-2-ylthiomethyl)-succinyl]-L-p-henylalanine-N-methylamidecarboxyalkylamino-based compounds; chelators (EDTA, cysteine, acetylcysteine, D-penicillamine, and gold salts); bis(dioxopiperzaine); NEOVASTAT; KB-R7785; ILOMASTAT; RPR-122818; SOLIMASTAT; BB-1101; BB-2983; BB-3644; BMS-275291; D-1927, D-5410; CH-5902, CH-138; CMT-3; DERMOSTAT; DAC-MMPI; RS-1130830 and RS-113-080; GM-1339; GI-155704A; ONO-4817; AG-3433, AG-3088, PRINOMASTAT; CP-544439; POL-641: SC-964; SD-2590; PNU-142769; SU-5402; PGE-2946979, PGE-4304887; fibrolase-conjugate; EF-13; S-3304; CGS-25015 and CGS-27023A; XR-168; RO1130830; D-9120; BB-2827; BB-1101 (2S-allyl-N1-hydroxy-3R-isobutyl-N4-(1S-methylcarbamoyl-2-phenylethyl)-su-ccinamide), BB-2983, solimastat (N′-[2,2-Dimethyl-[1(S)—[N-(2-pyridyl)carba-moyl]propyl]-N4-hydroxy-2(R)-isobutyl-3(S)-methoxysuccinamide), N4-hydroxy-N1-[2-(methylamino)-2-oxo-1-(phenylmethyl)ethyl]-2-(2-methylpr-opyl)-3-[(2-thienylthio)methyl]-, [2R-[1 (S*),2R*,3 S*]]-[CAS]), rebimastat (L-Valinamide, N—((2S)-2-mercapto-1-oxo-4-(3,4,4-trimethyl-2,5-dioxo-1-imidazolidinyl)butyl)-L-leucyl-N,3-dimethyl-[CAS]); PS-508; CH-715; nimesulide (Methanesulfonamide, N-(4-nitro-2-phenoxyphenyl)-[CAS]-), hexahydro-2-[2(R)-[1(RS)-(hydroxycarbamoyl)-4-phenylbutyl]nonanoyl]-N-(-2,2,6,6-etramethyl-4-piperidinyl)-3(S)-pyridazine carboxamide, Cipemastat (1-Piperidinebutanamide, .beta.-(cyclopentylmethyl)-N-hydroxy-Gamma-oxo-A-lpha-[(3,4,4-trimethyl-2,5-dioxo-1-imidazolidinyl)methyl]-,(AlphaR,.beta.R-)-[CAS]), 5-(4′-biphenyl)-5-[N-(4-nitrophenyl)piperazinyl]barbituric acid, 6-methoxy-1,2,3,4-tetrahydro-norharman-1-carboxylic acid, Ro-31-4724 (L-Alanine, N-[2-[2-(hydroxyamino)-2-oxoethyl]-4-methyl-1-oxopentyl]-L-le-ucyl-, ethyl ester[CAS]), N-hydroxy-2,2-dimethyl-4-((4-(4-pyridinyloxy)phe-nyl)sulfonyl)-, (3R)-[CAS]), PNU-142769 (2H-Isoindole-2-butanamide, 1,3-dihydro-N-hydroxy-Alpha-[(3S)-3-(2-methylpropyl)-2-oxo-1-(2-phenyleth-yl)-3-pyrrolidinyl]-1,3-dioxo-, (AlphaR)-[CAS]), (S)-1-[2-[[[(4,5-Dihydro-5-thioxo-1,3,4-thiadiazol-2-yl)amino]-carbonyl]amino]-1-oxo-3-(pentafluoro-phenyl)propyl]-4-(2-pyridinyl)piperazine, SC-77964, PNU-171829, N-hydroxy-2(R)-[(4-methoxybenzene-sulfonyl)(4-picolyl)amino]-2-(2-tetrahy-drofuranyl)-acetamide, L-758354 ((1,1′-Biphenyl)-4-hexanoic acid, Alpha-butyl-Gamma-(((2,2-dimethyl-1-((methylamino)carbonyl)propyl)amino)c-arbonyl)-4′-fluoro-, (AlphaS-(AlphaR*,GammaS*(R*)))-[CAS]); antibodies; and analogues or derivatives thereof.

7. A method according to claim 5 wherein the MMPI is selected from the group consisting of an MMP-2 inhibitor, an MMP-9 inhibitor and an MMP-2/9 inhibitor.

8. A method according to claim 4 wherein the inhibitor is Ilomastat (GM6001).

9. A method according to claim 4 wherein the inhibitor is ((2R)-[(4-Biphenylylsulfonyl)amino]-N-hydroxyl-3-phenylpropionamide).

10. The method of claim 1 wherein the inhibitor is locally administered.

11. The method of claim 9 wherein the inhibitor is delivered via a controlled release device.

12. The method of claim 10 wherein the controlled release device is implanted in the eye.

13. The method of claim 9 wherein the inhibitor is delivered by injection.

14. The method of claim 9 wherein the inhibitor is administered as an ophthalmic solution, gel or cream.

15. The method of claim 1 wherein the inhibitor is administered in a dose range of about 0.1 μg/ml to about 500 μg/ml.

16. The method of claim 15 wherein the inhibitor is administered in a dose range of about 1 μg/ml to 100 μg/ml.

17. The method of claim 16 wherein the inhibitor is administered in a dose range of about 5 μg/ml to 25 μg/ml.

18. The method of claim 1 wherein the inhibitor is administered in combination with at least one additional MMP inhibitor.

19. The method of claim 1 wherein the inhibitor is administered with another pharmacologically active agent.

20. A composition for the treatment or prevention of cataract comprising an MMPI and a pharmaceutically acceptable carrier.

21. A composition according to claim 21, wherein the MMPI is wherein the MMPI is selected from the group consisting of: ((2R)-[(4-Biphenylylsulfonyl)amino]-N-hydroxyl-3-phenylpropionamide); actinonin (3-[[1-[[2-(hydroxymethyl)-1-pyrrolidinyl]carbamoyl]-octano-hydroxamic acid); bromocyclic-adenosine monophosphate; N-chlorotaurine; BATIMISTAT (BB-94); CT1166 (N1 {N-[2-(morpholinosulphonylamino)-ethyl]-3-cyclohexyl-2-(S)-propanamidyl}-N4-hydroxy-2-(R)-[3-(4-methylphenyl)propyl]-succinamide); estramustine (estradiol-3-bis(2-chloroethyl)carbamat-e); eicosa-pentaenoic acid; MARIMASTAT (BB-2516); matlystatin-B; peptidyl hydroxamic acids; N-phosphonalkyl dipeptides; protocatechuic aldehyde (3,4-dihydroxybenzaldehyde); Ro-31-7467 (2-[(5-bromo-2,3-dihydro-6-hydroxy-1,3-dioxo-1H-benz[de]isoquinol-in-2-yl)methyl](hydroxy)-[phosphinyl]-N-(2-oxo-3-azacyclotridecanyl)-4-met-hylvaleramide); tetracyclines; 1,10-phenanthroline (o-phenanthroline[4-(N-hydroxyamino)-2R-isobutyl-3S-(thiopen-2-ylthiomethyl)-succinyl]-L-p-henylalanine-N-methylamidecarboxyalkylamino-based compounds; chelators (EDTA, cysteine, acetylcysteine, D-penicillamine, and gold salts); bis(dioxopiperzaine); NEOVASTAT; KB-R7785; ILOMASTAT; RPR-122818; SOLIMASTAT; BB-1101; BB-2983; BB-3644; BMS-275291; D-1927, D-5410; CH-5902, CH-138; CMT-3; DERMOSTAT; DAC-MMPI; RS-1130830 and RS-113-080; GM-1339; GI-155704A; ONO-4817; AG-3433, AG-3088, PRINOMASTAT; CP-544439; POL-641: SC-964; SD-2590; PNU-142769; SU-5402; PGE-2946979, PGE-4304887; fibrolase-conjugate; EF-13; S-3304; CGS-25015 and CGS-27023A; XR-168; RO1130830; D-9120; BB-2827; BB-1101 (2S-allyl-N1-hydroxy-3R-isobutyl-N4-(1S-methylcarbamoyl-2-phenylethyl)-su-ccinamide), BB-2983, solimastat (N′-[2,2-Dimethyl-[(S)-[N-(2-pyridyl)carba-moyl]propyl]-N4-hydroxy-2(R)-isobutyl-3(S)-methoxysuccinamide), N4-hydroxy-N1-[2-(methylamino)-2-oxo-1-(phenylmethyl)ethyl]-2-(2-methylpr-opyl)-3-[(2-thienylthio)methyl]-, [2R-[1 (S*),2R*,3S*]]-[CAS]), rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-[CAS]); PS-508; CH-715; nimesulide (Methanesulfonamide, N-(4-nitro-2-phenoxyphenyl)-[CAS]-), hexahydro-2-[2(R)-[1(RS)-(hydroxycarbamoyl)-4-phenylbutyl]nonanoyl]-N-(-2,2,6,6-etramethyl-4-piperidinyl)-3(S)-pyridazine carboxamide, Cipemastat (1-Piperidinebutanamide, .beta.-(cyclopentylmethyl)-N-hydroxy-Gamma-oxo-A-lpha-[(3,4,4-trimethyl-2,5-dioxo-1-imidazolidinyl)methyl]-,(AlphaR,.beta.R—)-[CAS]), 5-(4′-biphenyl)-5-[N-(4-nitrophenyl)piperazinyl]barbituric acid, 6-methoxy-1,2,3,4-tetrahydro-norharman-1-carboxylic acid, Ro-31-4724 (L-Alanine, N-[2-[2-(hydroxyamino)-2-oxoethyl]-4-methyl-1-oxopentyl]-L-le-ucyl-, ethyl ester[CAS]), N-hydroxy-2,2-dimethyl-4-((4-(4-pyridinyloxy)phe-nyl)sulfonyl)-, (3R)-[CAS]), PNU-142769 (2H-Isoindole-2-butanamide, 1,3-dihydro-N-hydroxy-Alpha-[(3S)-3-(2-methylpropyl)-2-oxo-1-(2-phenyleth-yl)-3-pyrrolidinyl]-1,3-dioxo-, (AlphaR)-[CAS]), (S)-1-[2-[[[(4,5-Dihydro-5-thioxo-1,3,4-thiadiazol-2-yl)amino]-carbonyl]amino]-1-oxo-3-(pentafluoro-phenyl)propyl]-4-(2-pyridinyl)piperazine, SC-77964, PNU-171829, N-hydroxy-2(R)-[(4-methoxybenzene-sulfonyl)(4-picolyl)amino]-2-(2-tetrahy-drofuranyl)-acetamide, L-758354 ((1,1′-Biphenyl)-4-hexanoic acid, Alpha-butyl-Gamma-(((2,2-dimethyl-1-((methylamino)carbonyl)propyl)amino)c-arbonyl)-4′-fluoro-, (AlphaS-(AlphaR*,GammaS*(R*)))-[CAS]); antibodies; and analogues or derivatives thereof.

22. A composition according to claim 21 wherein the MMPI is selected from the group consisting of an MMP-2 inhibitor, an MMP-9 inhibitor and an MMP-2/9 inhibitor.

23. A composition according to claim 21 wherein the inhibitor is Ilomastat (GM6001).

24. A composition according to claim 21 wherein the inhibitor is ((2R)-[(4-Biphenylylsulfonyl)amino]-N-hydroxyl-3-phenylpropionamide).

25.-28. (canceled)

29. A delivery device for the administration of a matrix metalloproteinase inhibitor to a region of an eye of a patient.

30. A delivery device according to claim 29 wherein the device is a contact lens.

31. A delivery device according to claim 29 wherein the device is an intra-ocular lens.
